(19) United States
(12) Patent Application Publication
(10) Pub. No.: US 2020/0367813 A1
(43) Pub. Date: Nov. 26, 2020

(54) FACE-RECOGNIZABLE WALL-MOUNTED ALCOHOL DETECTOR

(71) Applicant: Shenzhen Well Electric Co., Ltd., Shenzhen (CN)

(72) Inventors: Zhi Chen, Shenzhen (CN); Yung Chan, Shenzhen (CN); Dong Chen, Shenzhen (CN); Rong Pan, Shenzhen (CN)

(21) Appl. No.: 16/659,570

(22) Filed: Oct. 22, 2019

(30) Foreign Application Priority Data
May 20, 2019 (CN) 201910419847.6
May 29, 2019 (CN) 201910458573.1

(51) Int. Cl. A61B 5/00; A61B 5/08; A61B 5/097; A61B 5/1171

(52) U.S. Cl. CPC A61B 5/4845; A61B 5/082; A61B 5/6889; A61B 5/1176; A61B 5/097

(57) ABSTRACT

The present application relates to the field of alcohol detection. The face-recognizable wall-mounted alcohol detector according to the present application comprises an enclosure, an alcohol detecting module, and a breathing cup. The enclosure is provided with a receiving chamber therein, and has a first side and a second side opposite to each other, the first side having a connection opening in communication with the receiving chamber, an air venting passage being provided on the side wall of the connection opening, and the second side having a dock interface in communication with the receiving chamber; the alcohol detecting module has a case on which an air detection inlet is provided on the case of the alcohol detecting module; the breathing cup has an air outlet at one end, a breathing opening at the other end, and an air vent on the side wall thereof.

### CROSS REFERENCE TO RELATED APPLICATIONS

The present application claims the priority to Chinese Patent Application No. 201910458573.1 filed on May 29, 2019, which is incorporated herein by reference in its entirety for all purposes.

### TECHNICAL FIELD

The present application relates to the field of alcohol detection, and, in particular, to a face-recognizable wall-mounted alcohol detector.

### BACKGROUND

An alcohol detector is an apparatus used for detecting whether alcohol is taken in a human body and how much alcohol is taken in. It can be used as a tool for detecting how much alcohol a driver has drunk for a traffic policeman on duty, so as to effectively reduce the occurrence of significant traffic accidents. An alcohol detector can also be used to detect the level of alcohol in the air expired from a human body on other occasions, for example, at some high-risk firms where it is prohibited to be on duty after drinking, so as to avoid personal casualties and significant property losses.

Conventional alcohol detectors usually comprise an alcohol detecting module and a breathing cup. During use, a subject breathes into the breathing cup with the mouth aiming at the cup, so that the alcohol detecting module can detect the air exhaled by the subject and measure the level of alcohol in the exhaled air.

On some occasions, if there is a large population of subjects, the subjects have to line up and sequentially be subject to the alcohol detector for detection. When the former subject leaves and the next one is tested, the air exhaled by the former remains in the breathing cup, which is not hygienic and will degrade the use experience of the next subject, and the air remained in the breathing cup may interfere with the detection result of the next subject.

In addition, the conventional alcohol detectors comprise an enclosure in which the alcohol detecting module is usually fixed. As a result, when the alcohol detecting module is damaged, it is inconvenient to repair, and sometimes it even causes the alcohol detector to be discarded as a whole, resulting in higher maintenance cost.

### SUMMARY

In view of the above problems, the present invention provides a face-recognizable wall-mounted alcohol detector, one main object of which is to address the technical problems present in the conventional design of an alcohol detector, that is, unhygiene and inconvenient maintenance when the alcohol detecting module is damaged.

The present application mainly provides the following technical solutions to achieve the above object:

One embodiment of the present application provides a face-recognizable wall-mounted alcohol detector, comprising an enclosure, an alcohol detecting module, and a breathing cup;

the enclosure is provided with a receiving chamber therein, and has a first side and a second side opposite to each other, the first side having a connection opening in communication with the receiving chamber, an air venting passage being provided on the side wall of the connection opening, and the second side having a dock interface in communication with the receiving chamber;

the alcohol detecting module has a case on which an air detection inlet is provided, and is removably inserted into the receiving chamber vie the dock interface through the case;

the breathing cup has an air outlet at one end, a breathing opening at the other end, and an air vent on the side wall thereof, the breathing cup being adapted for being inserted into the connection opening and being in communication with the air detection inlet via the air outlet; wherein, when the breathing cup is inserted into the connection opening, the air vent is in communication with the air venting passage.

In a further embodiment of the present application, the alcohol detecting module comprises an air mouthpiece, a recess is provided on the case, the opening of which is opposite to the connection opening, and one end of the air mouthpiece has an air detection inlet and is protruded from the bottom of the recess;

the breathing cup is adapted for being inserted into the recess via the connection opening, and snap connected in the recess;

one end of the air mouthpiece is adapted for being inserted into the air outlet of the breathing cup when the breathing cup is inserted into the recess, so as to be in communication with the inside of the breathing cup.

In a further embodiment of the present application, a snap-in groove is provided on one end of the breathing cup, and a raised snap bar is provided on the side wall of the recess;

when one end of the breathing cup is inserted into the recess, the snap bar is snap connected in the snap-in groove.

In a further embodiment of the present application, a tubular protrusion having the air vent is provided on the side wall of the breathing cup.

In a further embodiment of the present application, the enclosure comprises a first half enclosure body having a first portion of the receiving chamber, and a second half enclosure body having the second portion of the receiving chamber; the first half enclosure body and the second half enclosure body are adapted for being snap fit together, so that the first portion and the second portion fit together to form the receiving chamber;

in particular, the connection opening is provided on the first half enclosure body and in communication with the receiving chamber via the first portion; and the dock interface is provided on the second half enclosure body and in communication with the receiving chamber via the second portion.

In a further embodiment of the present application, a limiting plate is provided inside the first half enclosure body;

the limiting plate is provided in plurality and at intervals, and all the limiting plates cooperate with each other to confine the first portion of the receiving chamber.

In a further embodiment of the present application, the side of one end of the limiting plate departing from the first half enclosure body is provided with a chamfer, which is the side of the limiting plate adjacent to the first portion.

In a further embodiment of the present application, a receiving hole acting as the second portion is provided on the second half enclosure body;

a convex rib is provided on the inner wall of the receiving hole for supporting the case of the alcohol detecting module.

In a further embodiment of the present application, the alcohol detector further comprises a cover body;

the cover body removably covers the dock interface.

By adopting the above technical solutions, the face-recognizable wall-mounted alcohol detector according to the present application has at least the following beneficial effects:

in the technical solutions provided by the present application, an air vent is provided on the side wall of the breathing cup, which, when the breathing cup is connected to the air detection inlet of the alcohol detecting module, is in communication with the air venting passage on the enclosure, so that when a subject breathes into the breathing cup, excess air which does not enter the air detection inlet can escape from the air venting passage, so as to reduce the air remained in the breathing cup, improving the use experience of the next subject which breathes into the breathing cup; and

in addition, the alcohol detecting module can be removed for repairing or replacing since it can be removed from the inside of the receiving chamber, facilitating better maintenance.

The above summary merely provides a general description on the technical solutions of the present application, for the purpose of better understanding the technical means that can be adopted by the present application and practiced according to the disclosure of the present application. The preferred embodiments of the present application are described below in detail in combination with the drawings.

### BRIEF DESCRIPTION OF DRAWINGS

FIG. 1 is an schematically structural and partially exploded diagram of a face-recognizable wall-mounted alcohol detector according to one embodiment of the present application;

FIG. 2 is an assembled structure diagram of a face-recognizable wall-mounted alcohol detector according to one embodiment of the present application;

FIG. 3 is the structural diagram of a breathing cup according to one embodiment of the present application when viewed from the first visual angle;

FIG. 4 is the structural diagram of a breathing cup according to one embodiment of the present application when viewed from the second visual angle;

FIG. 5 is the schematic structural diagram of an alcohol detecting module according to one embodiment of the present application;

FIG. 6 is the schematic structural diagram of a first half enclosure body according to one embodiment of the present application;

FIG. 7 is the schematic structural diagram of the back of a second half enclosure body according to one embodiment of the present application; and

FIG. 8 is the schematic structural diagram of the front of a second half enclosure body according to one embodiment of the present application.

### REFERENCE NUMBERS

1. enclosure 2. alcohol detecting module; 3. breathing cup; 4. cover body: 5. air mouthpiece; 6. transparent piece; 7. camera; 8. connecting tube; 11. first half enclosure body; 12. second half enclosure body; 21. case: 31. tubular protrusion; 51. air detection inlet: 101. connection opening; 102. air venting passage; 111. limiting plate: 112. male plug 120. receiving hole; 121. dock interface; 122. convex rib; 123. hanging hole; 211. recess: 212. snap bar: 213. female socket; 301. breathing opening; 302. air vent; 303. snap-in groove; 304. air outlet; 1111. chamfer; 100. alcohol detector.

### DETAILED DESCRIPTION

The technical solutions defined in the embodiments of the application will be clearly and completely described below in combination with the drawings. It is to be noted that, if there are directional indications (for example, on/above, under/below, left, right, front, back . . . ) involved in the embodiments of the present application, the directional indications are merely intended for explaining the relative position relationship or moving relationship between components at some specific postures (for example, as shown in the figures), and if the specific postures are changed, these directional indications will be correspondingly changed therewith. In addition, if the expressions “first”, “second” or the like are involved in the embodiments of the present application, they are merely intended for the purpose of description, and should not be construed as indicating or implying relative importance or the number of the technical features as mentioned. However, it can be indicated or implied that the technical features that are defined as “first”, “second” or the like comprise at least one of such technical feature.

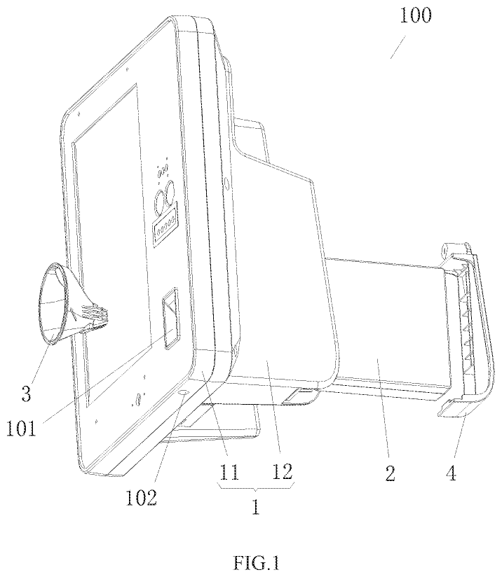

As shown in FIGS. 1-8, a face-recognizable wall-mounted alcohol detector 100 according to one embodiment of the present application comprises an enclosure 1, an alcohol detecting module 2 and a breathing cup 3. A receiving chamber is provided in the enclosure 1. The enclosure 1 has a first side and a second side opposite to each other. The first side has a connection opening 101 in communication with the receiving chamber. The side wall of the connection opening 101 is provided with an air venting passage 102. The second side is provided with a dock interface 121. In a particular application example, the first side can be the front side of the alcohol detector 100, and the second side can be the back side of the alcohol detector 100.

As shown in FIG. 5, the above alcohol detecting module 2 has a case 21. An air detection inlet 51 is provided on the case 21. the alcohol detecting module 2 is removably inserted into the receiving chamber via the dock interface 121 through the case 21. When the alcohol detecting module 2 fails, the alcohol detecting module 2 can be immediately removed for repairing or replacing, facilitating better maintenance, since the alcohol detecting module 2 can be removed from the receiving chamber.

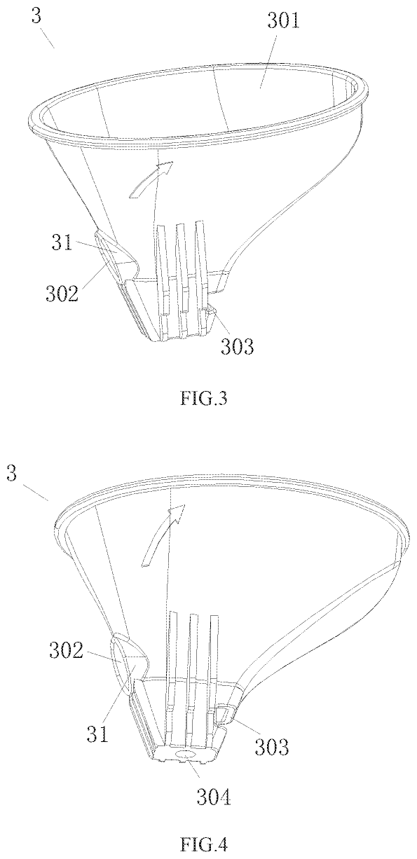

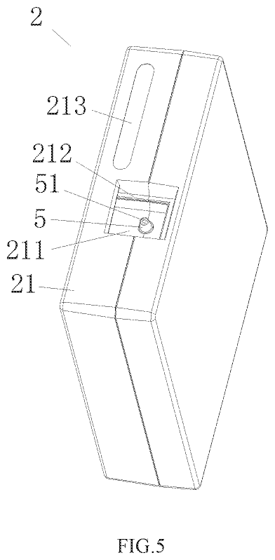

As shown in FIGS. 3 and 4, an air outlet 304 is provided at one end of the breathing cup 3, a blowing opening 301 is provided the other end of the breathing cup 3, and an air vent 302 is provided on the side wall. The breathing cup 3 is adapted for being inserted in the connection opening 101, and being in communication with the air detection inlet 51 via the air outlet 304. In particular, when the breathing cup 3 is inserted into the connection opening 101, the air vent 302 is in communication with the air venting passage 102. In a particular application example, the above air outlet 304 can be a 0-type outlet.

During using the above alcohol detector 100, a subject breathes into the breathing cup 3 via the breathing opening 301, air enters the alcohol detecting module 2 from the air detection inlet 51, and the received air is detected by the alcohol detecting module 2 regarding the level of the alcohol. In particular, the excess air which does not enter the air detection inlet 51 is escaped from the air venting passage 102 via the air vent 302 on the breathing cup 3, so as to prevent air from being remained in the breathing cup 3, improving the use experience of the subject when breathing into the breathing cup 3.

It is to be noted that, the above alcohol detector 100 can be breathed into by a plurality of breathing manners. For example, a subject can directly breathe into the breathing cup 3 with the mouth aiming the breathing cup 3, or can breathe into the breathing cup 3 via an air tube by inserting one end of the air tube into the breathing cup 3. When a subject breathes into the breathing cup 3 by using an air tube, the air tube can be a straw or a disposal air tube used by the subject himself when drinking a beverage, so as to be cleaner and more hygienic.

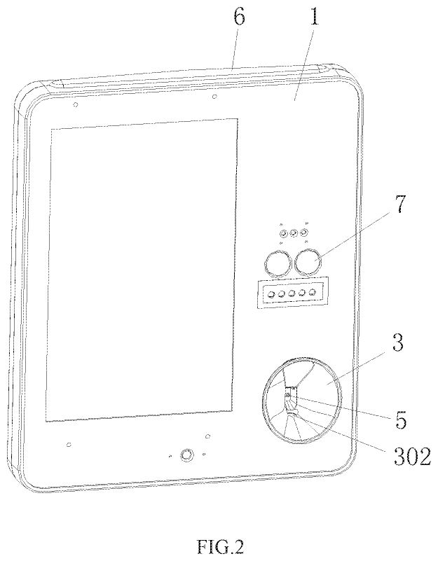

As shown in FIGS. 2 and 5, the above alcohol detecting module 2 can comprise air mouthpiece 5. A recess 211 is provided on the case 21. The opening of the recess 211 is opposite to the connection opening 101 on the enclosure 1. One end of the air mouthpiece 5 is provided with the air detection inlet 51, and protruded from the bottom of the recess 211. The breathing cup 3 is adapted for being inserted into the recess 211 via the connection opening 101, and snap connected with the recess 211. One end of the mouth piece 5 is adapted for being inserted into the air outlet 304 of the breathing cup 3 when the breathing cup 3 is inserted into the recess 211, so as to be communication with the inside of the breathing cup 3.

By adopting the above configuration, the connection opening 101 on the enclosure 1 acts as a position-dodging opening, that is, when one end of the breathing cup 3 is inserted into the recess 211 on the case 21, the air mouthpiece 5 in the recess 211 is inserted into the air outlet 304 on the breathing cup 3, so that the parts of both the breathing cup 3 and the alcohol detecting module 2 are inserted into the inside of one another, ensuring a better connection therebetween and thus avoiding loosening.

In a particular application example, the above case 21 can be formed by snap connecting two half cases followed by fixing with a screw. On some occasions, the two half cases can be called as the half top case and the half bottom case, respectively. The above recess 211 is formed by combining the half recess on the half top case and the half recess on the half bottom case.

Further, as shown in FIGS. 3-5, one end of the breathing cup 3 is provided with a snap-in groove 303. A raised snap bar 212 is provided on the side wall of the recess 211. When one end of the breathing cup 3 is inserted into the recess 211, the snap bar 212 is snapped into the snap-in groove 303, so as to achieve snap-fit connection between the breathing cup 3 and the recess 211.

Further, as shown in FIG. 3, a tubular protrusion 31 having the air vent 302 can be provided on the side wall of the breathing cup 3. In this example, the providing of the tubular protrusion 31 has the effect of facilitating the communication between the air vent 302 and the air venting passage 102.

Further, as shown in FIG. 3, an reinforcement rib can be provided on the side wall of the breathing cup 3, so as to reinforce the structural strength of the breathing cup 3, and serve the functions of fool-proofing and position limiting.

As shown in FIG. 1, the enclosure 1 comprises a first half enclosure body 11 and the second half enclosure body 12. The first half enclosure body 11 has the first portion of the receiving chamber, and the second half enclosure body has the second portion of the receiving chamber. Both the first half enclosure body and the second half enclosure body 12 are adapted for being snap fit with each other, so that the first portion and the second portion fit with each other to form the receiving chamber. In particular, the connection opening 101 is provided on the first half enclosure body 11, and in communication with the receiving chamber via the first portion. The dock interface 121 is provided on the second half enclosure body 12, and in communication with the receiving chamber via the second portion. In order to improve fixing effect, the first half enclosure body 11 and the second half enclosure body 12 can be further secured via a screw.

In the above example, dividing the enclosure 1 into the first half enclosure body 11 and the second half enclosure body 12 provides the effect of convenient processing and assembling.

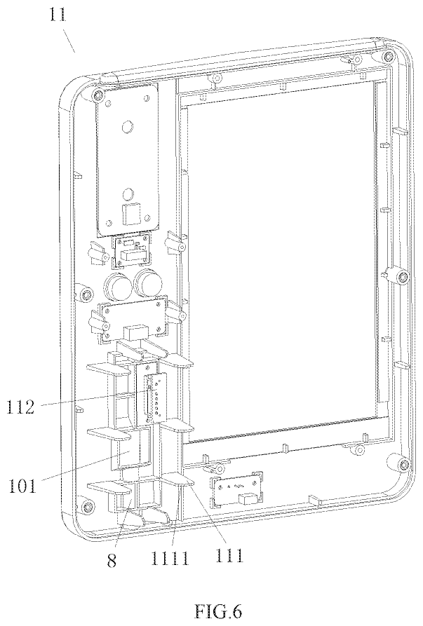

Further, as shown in FIG. 6, the enclosure 1 can be integrally formed with a connecting tube 8, inside which the air venting passage 102 is provided. One end of the air venting passage 8 extends through the side wall of the connection opening 101, and the other end thereof extends through the side wall of the enclosure 1. Preferably, the connecting tube 8 is integrally formed on the first half enclosure body 11 and the other end of the connecting tube 8 extends through the side wall of the first half enclosure body 11.

In the above example, since the connecting tube 8 is integrally formed on the enclosure 1, the connection therebetween has a better stability. In addition, since the connecting tube 8 extends through the side wall of the enclosure 1, the air remained in the breathing cup 3 can be guided out from the side, so as to avoid flowing back onto the face of a subject and degrading the use experience of the subject.

Further, as shown in FIG. 6, the inside of the first half enclosure body 11 is provided with a limiting plate 111, which can be provided in plurality and at intervals. In particular, all the limiting plates 111 cooperate with each other to form the first portion of the receiving chamber.

By adopting the above configuration, the limiting plate 111 can, on one hand, limit the position of the alcohol detecting module 2, and, on the other hand, serve the function of a reinforcement rib, improving the structural strength of the first half enclosure body 11. In addition, in comparison with providing a limiting groove or the like on the first half enclosure body 11, the limiting plate 11 occupies smaller volume, and only a few limiting plates 11 can achieve the function of limiting position of the case 21 of the alcohol detecting module 2, thereby providing the effect of simplifying the structure of the first half enclosure body 11 and reducing the processing cost.

As shown in FIG. 6, the side of the end of the limiting plate 111 departing from the first half enclosure body 111 can be provided with a chamfer 1111. In particular, this side is the side of the limiting plate 111 adjacent to the first portion. Particularly, the chamfer 1111 in this example has the effect of guiding the inserting of the case 21 of the alcohol detecting module 2 into the first portion.

It is to be noted here that, the particular form of the chamfer 1111 is not limited, for example, it can be a rounded angle or oblique angle. Preferably, the chamfer 1111 is an oblique angle, so that the case 21 of the alcohol detecting module 2 can be quickly inserted into the first portion of the receiving chamber.

In order to fix the alcohol detecting module 2, a female socket 213 can be provided on the case 21 of the alcohol detecting module 2, and a male plug 112 can be provided on the inside of the first half enclosure body 11. When the alcohol detecting module 2 is inserted into the enclosure 1, the male plug 112 is inserted into the female socket 213. In a particular application example, the male plug can be PCB male plug, and, correspondingly, the female socket can be a PCB female socket.

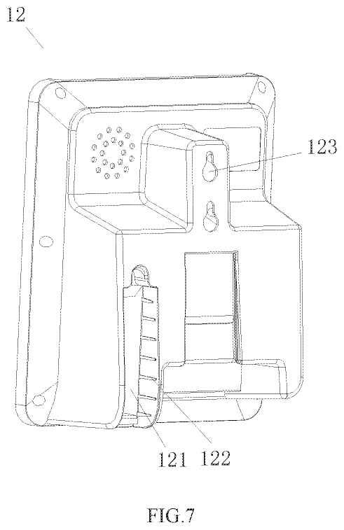

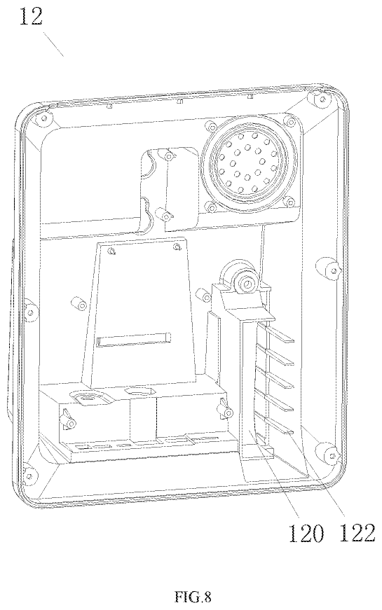

Further, as shown in FIGS. 7 and 8, a receiving hole 120 acting as the second portion can be provided on the second half enclosure body 12. A convex rib 122 can be provided on the inner wall of the receiving hole 120. The convex rib 122 is used to support the case 21 of the alcohol detecting module 2. In particular, the structure of the receiving hole 120 is convenient for processing.

The convex ribs 122 can have a number of 2 or more, and can be sequentially provided at intervals, so as to facilitate the assembling and limiting of the alcohol detecting module 2.

Further, as shown in FIG. 1, the face-recognizable wall-mounted alcohol detector 100 according to the present application can further comprise a cover body 4. The cover body 4 can removably cover the dock interface 121, so as to protect the alcohol detecting module 2.

In a particular application example, the cover body 4 can be snap connected at the dock interface 121, and connected to the second half enclosure body 12 via a screw, so as to facilitate the removing of the cover body 4 and more stable covering of the cover body 4.

As shown in FIG. 7, a hanging hole 123 can be provided on the second half enclosure body 12, through which the face-recognizable wall-mounted alcohol detector 100 according to the present application can be hanged on a wall for use.

As shown in FIG. 2, the alcohol detector 100 according to the present application can further comprises a camera 7, which is provided in the enclosure 1, and can be seen via an opening on the enclosure 1. In one particular application example, the number of the cameras 7 is two.

As shown in FIG. 2, a lamp body, for example, an LED lamp, can be provided in the enclosure 1. A mounting hole is provided on the enclosure 1, and a transparent piece 6 is inserted in the mounting hole. In particular, the mounting hole can be an indicator light hole, and the transparent piece 6 can be the indicator light cover, which can be made of transparent plastics. When the alcohol detector 100 according to the present application is operating, the light emitted from the light body irradiates onto the transparent piece 6 to cause the latter to emit light, thereby providing the effect of indicating that the alcohol detector 100 is working.

The mounting hole can be formed by snap fitting the first half hole on the first half enclosure body 11 with the second half hole on the second half enclosure body 12. In one particular application example, the transparent piece 6 can assume a long strip shape, and be provided on top of the enclosure 1.

It is to be noted that, in the absence of conflict, the relevant technical features in the above examples can be combined with each other by those skilled in the art depending on actual needs, so as to achieve corresponding technical effects, the particular combinations of which are not provided here in details.

What is provided above is merely the preferred embodiments according to the present application, and the protection scope of the present application is not limited to the above embodiments. On the contrary, all the technical solutions obtained based on the concepts of the present application should fall within the protection scope of the present application. It should be noted that, for those skilled in the art, some improvements and modifications can be made without departing from the principles of the present applications, which should be also considered as falling within the protection scope of the present application.

What is claimed is:

1. A face-recognizable wall-mounted alcohol detector comprising an enclosure (1), an alcohol detecting module (2), and a breathing cup (3);
wherein the enclosure (1) is provided with a receiving chamber therein, and has a first side and a second side opposite to each other, the first side having a connection opening (101) in communication with the receiving chamber, an air venting passage (102) being provided on the side wall of the connection opening (101), and the second side having a dock inter face (121) in communication with the receiving chamber;
wherein the alcohol detecting module (2) has a case (21) on which an air detection inlet (51) is provided, and is removably inserted into the receiving chamber vie the dock interface (121) through the case (21); and
wherein the breathing cup (3) has an air outlet (304) at one end, a breathing opening (301) at the other end, and an air vent (302) on the side wall thereof, the breathing cup (3) being adapted for being inserted into the connection opening (101) and being in communication with the air detection inlet (51) via the air outlet (304); wherein, when the breathing cup (3) is inserted into the connection opening (101), the air vent (302) is in communication with the air venting passage (102).

2. The face-recognizable wall-mounted alcohol detector according to claim 1,
wherein the alcohol detecting module (2) comprises an air mouthpiece (5), a recess (211) is provided on the case (21), the opening of which is opposite to the connection opening (101), and one end of the air mouthpiece (5) has the air detection inlet (51) and is protruded from the bottom of the recess (211);
the breathing cup (3) is adapted for being inserted into the recess (211) via the connection opening (101), and snap connected in the recess (211); and
one end of the air mouthpiece (5) is adapted for being inserted into the air outlet (304) of the breathing cup (3) when the breathing cup (3) is inserted into the recess (211), so as to be in communication with the inside of the breathing cup (3).

3. The face-recognizable wall-mounted alcohol detector according to claim 2,
wherein a snap-in groove (303) is provided on one end of the breathing cup (3), and a raised snap bar (212) is provided on the side wall of the recess (211);
when one end of the breathing cup (3) is inserted into the recess (211), the snap bar (212) is snap connected in the snap-in groove (303).

4. The face-recognizable wall-mounted alcohol detector according to claim 1,
wherein a tubular protrusion (31) having the air vent (302) is provided on the side wall of the breathing cup (3).

5. The face-recognizable wall-mounted alcohol detector according to claim 1,
wherein the enclosure (1) comprises a first half enclosure body (11) having a first portion of the receiving chamber, and a second half enclosure body (12) having the second portion of the receiving chamber;
the first half enclosure body (11) and the second half enclosure body (12) are adapted for being snap fit together, so that the first portion and the second portion fit together to form the receiving chamber;
wherein the connection opening (101) is provided on the first half enclosure body (11) and in communication with the receiving chamber via the first portion; and the dock inter face (121) is provided on the second half enclosure body (12) and in communication with the receiving chamber via the second portion.

6. The face-recognizable wall-mounted alcohol detector according to claim 5,
wherein a limiting plate (111) is provided inside the first half enclosure body (11);
the limiting plate (111) is provided in plurality and at intervals, and all the limiting plates (111) cooperate with each other to confine the first portion of the receiving chamber.

7. The face-recognizable wall-mounted alcohol detector according to claim 6,
wherein the side of one end of the limiting plates (111) departing from the first half enclosure body (11) is provided with a chamfer (1111), which is the side of the limiting plate (111) adjacent to the first portion.

8. The face-recognizable wall-mounted alcohol detector according to claim 5,
wherein a receiving hole (120) acting as the second portion is provided on the second half enclosure body (1);
a convex rib (122) is provided on the inner wall of the receiving hole (120) for supporting the case (21) of the alcohol detecting module (2).

9. The face-recognizable wall-mounted alcohol detector according to claim 1,
wherein the alcohol detector further comprises a cover body (4);
the cover body (4) removably covers the dock inter face (121).
